(19) United States
(12) Patent Application Publication
(10) Pub. No.: US 2024/0096468 A1
(43) Pub. Date: Mar. 21, 2024

(54) ELECTRONIC SYSTEM FOR WOUND IMAGE ANALYSIS AND COMMUNICATION

(71) Applicant: KEYSTONE PHARMACY, LLC, MADISON, MS (US)

(72) Inventor: JEFFREY CLARK, MADISON, MS (US)

(21) Appl. No.: 17/933,844

(22) Filed: Sep. 20, 2022

Related U.S. Application Data
(63) Continuation-in-part of application No. 16/404,126, filed on May 6, 2019, now abandoned.

(51) Int. Cl. G16H 20/10; G16H 10/60; G16H 30/20; G16H 50/20; G16H 80/00

(52) U.S. Cl. CPC G16H 20/10; G16H 10/60; G16H 30/20; G16H 50/20; G16H 80/00

(57) ABSTRACT

Disclosed are various embodiments for wound image analysis and communication. In one embodiment, at least one image of a wound and prescription information are received from a mobile device. A verification is performed on the prescription information based at least in part on an analysis of the at least one image. A user interface is generated showing the image(s), the prescription information, and a result of the verification.

### BACKGROUND

Wounds are a common medical problem, especially among patient populations including nursing home patients, diabetics, and others with poor circulation. Wounds are areas of skin or breaks in skin that are or could become colonized by harmful bacteria or fungi. Wounds are typically assessed by a wound care provider, which could include vascular surgeons, orthopedic surgeons, nurse practitioners, podiatrists, and others. The wound care provider upon assessment will determine a course of treatment, which can include prescribing products that are available from a specialty pharmacy. The specialty pharmacy can create custom preparations with antibiotic and/or antifungal ingredients. These preparations may include creams, gels, powders, and so forth for administration topically or in an aqueous solution for soaking the wound (e.g., in a footbath).

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1A is a schematic block diagram of a networked environment according to various embodiments of the present disclosure.

FIG. 1B shows one example of a wound ruler used in one or more embodiments of the present disclosure.

FIG. 2 is a flowchart illustrating one example of functionality implemented as portions of a provider application executed in a provider computing device in the networked environment of FIG. 1A according to various embodiments of the present disclosure.

FIGS. 3 and 4 are flowcharts illustrating examples of functionality implemented as portions of a wound care management service executed in a computing environment in the networked environment of FIG. 1A according to various embodiments of the present disclosure.

FIG. 5 is a schematic block diagram that provides one example illustration of a computing environment employed in the networked environment of FIG. 1A according to various embodiments of the present disclosure.

### DETAILED DESCRIPTION

The present disclosure relates to an electronic system for wound care management. Under a typical wound care treatment scenario, a patient visits a wound care provider who assesses the wound. The wound care provider determines a course of treatment for the wound, which may include prescribing medications. Although antibiotics and other medications for treating wounds may be taken orally in some cases, patients with wounds may have poor circulation or other conditions which make it difficult to effectively treat the wounds with oral medication. Consequently, wound treatment providers may prescribe topically applied medications or medications that are dissolved in an aqueous bath so that they may be absorbed in and around the wound area. These prescriptions may be customized for the individual patient and may be available only from a specialty or compounding pharmacy.

Under existing approaches, wound care providers or their administrative staff may call in or fax prescriptions to the pharmacy. This may happen periodically, such as once per day, rather than immediately after the patient is assessed. Thus, the periodic processing can delay fulfilment of the prescription. Furthermore, from the prescription itself, the pharmacy staff may have little knowledge of the state of the wound and the healing process. Moreover, patients may not comply with the treatment regimen, impeding the healing process. The pharmacy staff may need to call the patient to assess progress and whether refills are needed.

Also, wound care providers may not have an easy way to assess treatment progress for a wound. Some wound care providers may use their smartphone cameras to take pictures of wounds over several visits, but it may be difficult to organize these pictures. The smartphone may not integrate into any electronic medical record system, and further, storage of the images by a default camera program may not meet the requirements of the Health Insurance Portability and Accountability Act of 1996 (HIPAA).

Various embodiments of the present disclosure introduce an electronic system for wound care management that incorporates specialized computer applications for use by wound care providers, pharmacies, and patients to better achieve treatment objectives. Wound care providers can use this technology to capture and manage images of wounds and submit the images along with prescriptions to a pharmacy. The pharmacy receives the information through the system and fulfills the prescriptions. A patient component facilitates tracking of wound healing and patient compliance. Various components of the system provide for wound measurement and three-dimensional model generation using a mobile device camera. Machine learning can be applied to images to ascertain wound parameters and verify and/or suggest treatment modalities.

Technical advantages of the present disclosure include accurately and automatically determining from images dimensions of a wound that may otherwise be inaccurately reported, automatically assessing progress in wound healing by way of machine learning applied to a set of wound images, automatically launching a process for capturing and submitting wound images triggered by machine-readable codes on a wound ruler, and so forth. In the following discussion, a general description of the system and its components is provided, followed by a discussion of the operation of the same.

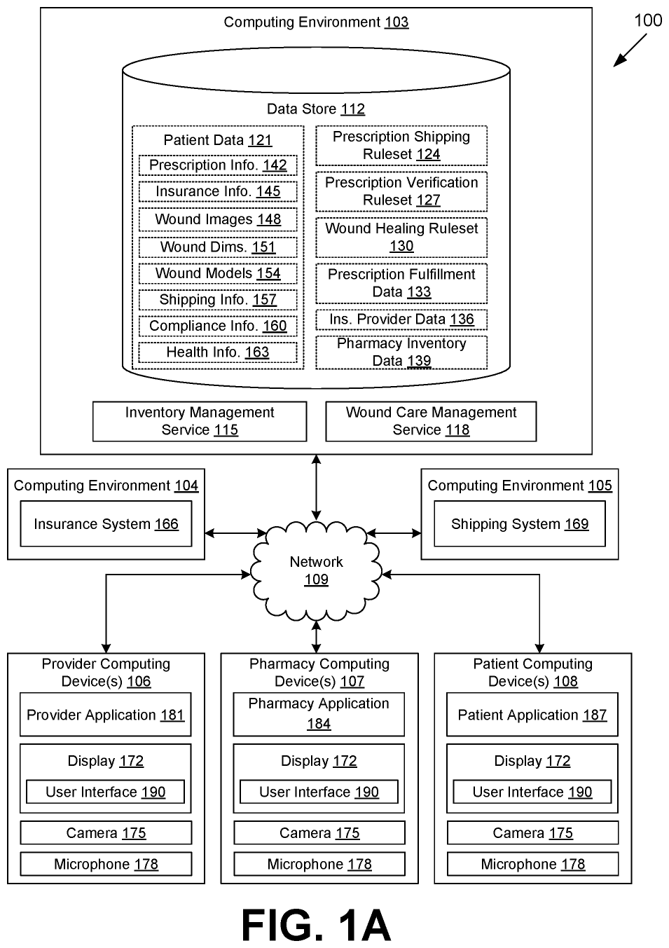

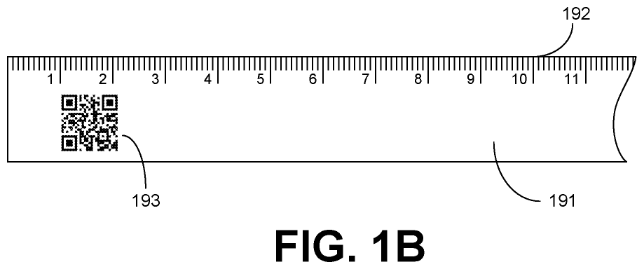

With reference to FIG. 1A, shown is a networked environment 100 according to various embodiments. The networked environment 100 includes a computing environment 103, a computing environment 104, a computing environment 105, one or more provider computing devices 106, one or more pharmacy computing devices 107, and one or more patient computing devices 108, which are in data communication with each other via a network 109. The network 109 includes, for example, the Internet, intranets, extranets, wide area networks (WANs), local area networks (LANs), wired networks, wireless networks, cable networks, satellite networks, or other suitable networks, etc., or any combination of two or more such networks.

The computing environments 103, 104, and 105 may comprise, for example, a server computer or any other system providing computing capability. Alternatively, the computing environments 103, 104, and 105 may employ a plurality of computing devices that may be arranged, for example, in one or more server banks or computer banks or other arrangements. Such computing devices may be located in a single installation or may be distributed among many different geographical locations. For example, the computing environments 103, 104, and 105 may include a plurality of computing devices that together may comprise a hosted computing resource, a grid computing resource, and/or any other distributed computing arrangement. In some cases, the computing environments 103, 104, and 105 may correspond to an elastic computing resource where the allotted capacity of processing, network, storage, or other computing-related resources may vary over time.

Various applications and/or other functionality may be executed in the computing environments 103, 104, and 105 according to various embodiments. Also, various data is stored in a data store 112 that is accessible to the computing environment 103. The data store 112 may be representative of a plurality of data stores 112 as can be appreciated. The data stored in the data store 112, for example, is associated with the operation of the various applications and/or functional entities described below.

The computing environment 103 may, for example, be operated by a pharmacy or a third-party provider of computer-based wound care management. The components executed on the computing environment 103, for example, include an inventory management service 115, a wound care management service 118, and other applications, services, processes, systems, engines, or functionality not discussed in detail herein. The inventory management service 115 is executed to track and manage the inventory of a pharmacy, including pharmaceutical products that are available to be used in fulfilling prescriptions to patients. The pharmacy may be a specialty or compounding pharmacy, where the pharmaceutical products may be mixed together and/or transformed from one form to another in order to fulfill a prescription.

The wound care management service 118 is executed to manage wound care for a patient beginning with an encounter of the patient with a wound care provider, fulfillment of a prescription for the wound, and tracking patient compliance and eventual healing of the wound. To this end, the wound care management service 118 may interface with computing devices associated with wound care providers, pharmacies, patients, insurance providers, shipping carriers, and/or other systems.

The data stored in the data store 112 includes, for example, patient data 121, a prescription shipping ruleset 124, a prescription verification ruleset 127, a wound healing ruleset 130, prescription fulfillment data 133, insurance provider data 136, pharmacy inventory data 139, and potentially other data. The patient data 121 includes data related to patients undergoing wound care. All of the patient data 121 is managed in accordance with applicable laws and regulations, including the Health Insurance Portability and Accountability Act of 1996 (HIPAA).

The patient data 121 includes prescription information 142, insurance information 145, wound images 148, wound dimensions 151, wound models 154, shipping information 157, compliance information 160, health information 163, and/or other data. The prescription information 142 includes information regarding a prescription that is supplied by an authorized wound care provider with prescribing authority, such as a physician, podiatrist, nurse practitioner, physician assistant, and so on, or his or her designees. Such information may include pharmaceutical names, including brand names or generic names, dosage, frequency of dose, a number of doses or length of time for the prescription, usage instructions, prescription date, expiration date, whether refills are permitted, prescriber's identifying information, patient's identifying information, and an electronic signature of the prescriber. In some cases, the prescription may include or relate to medical equipment.

The insurance information 145 describes one or more insurance policies that may be available to a patient. This can include insurance provider name, policy type, formularies, copayments, deductibles, out-of-pocket maximums, and/or other information that details how an insurance claim should be submitted, the patient's responsibility, and how much the insurance provider will pay.

The wound images 148 include one or more images of a patient's wound captured via a mobile device of the patient and/or wound care provider. The wound images 148 may be taken over time, beginning with an initial patient encounter and possibly including follow-up encounters and images captured by the patient. The wound images 148 may correspond to a video. In one embodiment, the user is directed to capture a video along a specified trajectory for the purpose of measuring a dimension of the wound and/or building a model of the wound.

The wound dimensions 151 correspond to dimensions of a patient's wound, including length, width, and depth, which may be manually measured by a patient or the wound care provider and entered via a user interface, or which may be automatically estimated through analysis of wound images 148. In particular, the automatic estimation of the wound's depth is especially helpful because, unlike wound length or width, manual measurement of depth may require insertion of a measuring device into the wound, which could interfere with healing or introduce further infection.

The wound models 154 correspond to two or three dimensional models of the wounds. For example, a user may be directed to capture multiple views or a video of the wound in the wound images 148. The wound images 148 can then be analyzed to extract parameters and generate a model (e.g., a wireframe) of the wound. The wound model 154 may be manipulated by a user to examine characteristics of the wound, including the wound dimensions 151.

The shipping information 157 includes information about packages sent to the patient to fulfill prescriptions. The shipping information 157 may include, for example, tracking information, shipping date, expected delivery date, special instructions for delivery, and/or other information. The compliance information 160 includes information about patient compliance with a prescription. For example, the compliance information 160 may schedule one or more manual or automated follow-ups with the patient to assess how well treatment of the wound is progressing and to ensure that the patient is using the prescription correctly.

The health information 163 may document health status for the patient. For example, the health information 163 may indicate that the patient has diabetes, is of advanced age, or is suffering from other conditions. The health information 163 may affect how the wound should be treated. To illustrate, a diabetic patient with a wound may suffer from poor circulation, and the wound may need to be soaked in order to deliver an antibiotic rather than delivering the antibiotic orally. Conversely, an elderly nursing home patient may benefit from topical preparations.

The prescription shipping ruleset 124 may control how prescriptions are shipped. For example, some prescriptions may need to be shipped with special handling, such as refrigeration or avoiding high temperatures. Some prescriptions may need signature confirmation, while others may be left in a mailbox or at a door. Because of the varying needs for the prescriptions, some prescriptions may be shipped using a subset of shipping carriers that are able to provide the required special handling or level of service.

The prescription verification ruleset 127 may control how prescriptions may be verified. For example, prescription information 142 may be cross-checked against health information 163 of the patient, wound dimensions 151, parameters extracted from wound images 148, and/or other information to determine whether the prescription information 142 is correct or may contain an error. The prescription verification ruleset 127 may be constructed with reference to processing on a set of past prescription information 142, past health information 163, and past wound images 148 via a machine learning approach. A set of past prescriptions may be verified and the corresponding patient information correlated with it to automatically determine types of prescriptions that correspond to types of patient health and types of wounds. Alternatively, the prescription verification ruleset 127 may be manually curated.

The wound healing ruleset 130 may be used to automatically determine whether a wound is healing at an expected rate. For example, a wound healing ruleset 130 may be applied to a sequence of wound images 148 taken at an interval to determine that the wound is healing as expected or is not healing as expected. The wound healing ruleset 130 may be generated automatically through a machine learning approach that compares reference wound images over time that correspond to proper healing (or conversely, lack of proper healing) to determine types of changes to the wounds that should be expected in view of treatment. A lack of expected healing may be used to send notifications to wound care providers or patients. In some cases, changes to the prescription may be automatically suggested based on an analysis of past decisions. For example, one antibiotic may be suggested to be substituted for another, or a different dosage of the same antibiotic may be suggested.

The prescription fulfillment data 133 tracks the progress of a prescription in its fulfillment. The prescription fulfillment data 133 may document statuses that a prescription has been received, that insurance approval is received, that the prescription has been verified, that the components prescribed are in stock, that the prescription has been prepared, that the prescription has been tendered to a shipping carrier, and the delivery status of the prescription with the shipping carrier.

The insurance provider data 136 includes information for verifying insurance coverage of patients and formularies. The pharmacy inventory data 139 includes data describing inventory for a pharmacy that may be used in fulfilling prescriptions, including prescriptions involving pharmaceuticals or medicines and medical equipment or devices. In some cases, the pharmacy inventory data 139 may indicate inventory status of components that may be mixed together in order to fulfill a prescription.

The computing environment 104 may be operated by an insurance provider. Among the components executed in the computing environment 104 is an insurance system 166. The insurance system 166 may support an application programming interface (API) for the submission of insurance payment requests and preauthorizations involved in fulfillment of prescriptions.

The computing environment 105 may be operated by a shipping carrier. Among the components executed in the computing environment 105 is a shipping system 169. The shipping system 169 may support APIs for the submission of shipping manifests and tracking queries for shipments of prescriptions being fulfilled by a pharmacy.

The provider computing devices 106, the pharmacy computing devices 107, and the patient computing devices 108 are representative of a plurality of client or server devices that may be coupled to the network 109. The provider computing devices 106, the pharmacy computing devices 107, and the patient computing devices 108 may comprise, for example, a processor-based system such as a computer system. Such a computer system may be embodied in the form of a desktop computer, a laptop computer, personal digital assistants, cellular telephones, smartphones, set-top boxes, music players, web pads, tablet computer systems, game consoles, electronic book readers, smartwatches, head mounted displays, voice interface devices, or other devices. The provider computing devices 106, the pharmacy computing devices 107, and the patient computing devices 108 may include a display 172. The display 172 may comprise, for example, one or more devices such as liquid crystal display (LCD) displays, gas plasma-based flat panel displays, organic light emitting diode (OLED) displays, electrophoretic ink (E ink) displays, LCD projectors, or other types of display devices, etc. The provider computing devices 106, the pharmacy computing devices 107, and the patient computing devices 108 may also include one or more cameras 175 and one or more microphones 178.

The provider computing devices 106, the pharmacy computing devices 107, and the patient computing devices 108 may be configured to execute various applications such as, respectively, a provider application 181, a pharmacy application 184, and a patient application 187. These applications may be executed, for example, to access network content served up by the computing environment 103 and/or other servers, thereby rendering a user interface 190 on the display 172. To this end, the applications may comprise, for example, a browser, a dedicated application, etc., and the user interface 190 may comprise a network page, an application screen, etc. The provider computing devices 106, the pharmacy computing devices 107, and the patient computing devices 108 may be configured to execute other applications such as, for example, medical record management applications, email applications, social networking applications, word processors, spreadsheets, and/or other applications.

Next, a general description of the operation of the various components of the networked environment 100 is provided. To begin, a wound care provider user interacts with a provider application 181 executed on a provider computing device 106 in order to enter prescription information 142 and/or other information for a patient undergoing treatment for a wound. The provider application 181 may enable the wound care provider user to take one or more wound images 148, which can include a video. The wound care provider user may measure the wound by sweeping the camera 175 along a specified trajectory. Further, such an approach may be applied to automatically determine the wound dimensions 151 and/or to generate the wound models 154.

The information corresponding to the prescription is submitted by the provider application 181 to the wound care management service 118. The wound care management service 118 may perform various processing on the information corresponding to the prescription. Such processing may include applying a prescription verification ruleset 127 in an automated way in order to verify whether the prescription information 142 is accurate. If verification problems are detected, the provider application 181 may render a user interface 190 informing the provider of the problem. Alternatively, the wound care management service 118 may send a notification to the provider (e.g., email, phone call, text message) descripting the detected verification issue. The provider user may then be given an opportunity to correct the issues via a user interface 190 or to dismiss any warnings.

The prescription information 142 and other information relating to the patient and prescription may then be made available to a pharmacy user via a pharmacy application 184 executed by the pharmacy computing device 107. For example, the pharmacy computing device 107 may render a user interface 190 that is a dashboard showing incoming prescriptions. The incoming prescriptions may be priority-ranked so that prescriptions for the most severe wounds are ranked with a higher priority to be fulfilled first. The priority ranking may also apply based on how the item is to be shipped and any unique shipping requirements for an item. For example, if a refrigerated truck is available only once daily at 2 pm for pickups, and a prescription is received at 1:30 for an item that must be refrigerated, a high priority may be assigned to the prescription so that it can be fulfilled by the pickup time.

Also, the user interface 190 of the pharmacy application 184 may highlight to the pharmacy user prescriptions for which the verification process has identified potential problems. A pharmacist or senior technician may review the prescription information 142 and/or other patient data 121 to assess the validity of the prescription. The pharmacy user may then contact the provider as needed to resolve any questions or raise concerns. A channel for initiating this communication may be provided via the wound care management service 118, the provider application 181, and the pharmacy application 184. The pharmacy application 184 may also communicate with the inventory management service 115 to identify to the pharmacy user in the user interface 190 any relevant information from the inventory, such as locations of items and/or low stock warnings.

As prescriptions are fulfilled, the pharmacy user may indicate fulfillment status via the user interface 190 of the pharmacy application 184. Status information may be sent back to the patient and/or the provider via the patient application 187 or the provider application 181, respectively. In addition, the pharmacy application 184 and/or the wound care management service 118 may communicate with the insurance system 166 to obtain authorizations or submit claims and with the shipping system 169 to enter shipping manifests for prescriptions. The wound care management service 118 may monitor the status of the fulfillment and send updates to the patient or provider, which can include insurance denials, insurance approvals, copayments required, shipment tracking information, estimated delivery date, and/or other information.

After the prescription is mailed to and received by the patient, the patient may be able to document the healing process via the patient application 187. For example, the patient may be asked to take one or more wound images 148 at periodic intervals via a camera 175 of the patient computing device 108 to determine the healing progress of the wound. The patient may also be invited to answer questions about the progress, such as compliance with taking or applying the prescribed items, any pain or other feelings about the wound, any smells, and/or any other information relevant to whether healing is occurring and whether the prescribed treatment plan is being complied with.

This information may be analyzed by the wound care management service 118. If expected healing is not occurring or if the wound is actually getting worse, pharmacy users and/or provider users may be notified. Suggested alternative prescriptions may be identified automatically. Depending on the healing progress as documented through the interaction with the patient application 187, refills of the prescription may be initiated or not initiated. For example, if the wound is nearly healed, the wound care management service 118 may deem refill of the prescription unnecessary. On the other hand, if the wound has not healed, the wound care management service 118 may automatically initiate a refilling of the prescription, potentially subject to confirmation by a user.

FIG. 1B shows one example of a wound ruler 191 used in one or more embodiments of the present disclosure. The wound ruler 191 may be made of plastic, wood, metal, or another material. The wound ruler 191 has one or more edges labeled with a measuring scale 192. For example, the measuring scale 192 may be labeled in centimeters, millimeters, inches, and/or other distance measures. The length of the wound ruler 191 may vary in different embodiments, but the wound ruler 191 is designed to be sufficiently long to place adjacent to a wound and facilitate measurement of the wound in an image captured of the wound. For example, the wound ruler 191 may be ten centimeters, twenty centimeters, thirty centimeters, or another length.

The wound ruler 191 may include a machine-readable code 193 visible on one or more sides of the wound ruler 191. The machine-readable code 193 may correspond to a quick-response (QR) code, a three-dimensional bar code, a two-dimensional bar code, or other code that is machine readable. When scanned by a patient computing device 108 (FIG. 1A), the machine-readable code 193 may cause an application to be launched for the purpose of capturing a wound image and communicating with a provider computing device 106 (FIG. 1A) and/or a pharmacy computing device 107 (FIG. 1A).

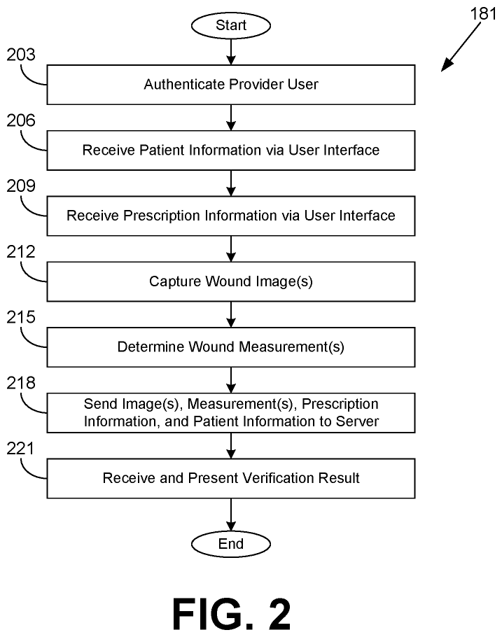

Referring next to FIG. 2, shown is a flowchart that provides one example of the operation of a portion of the provider application 181 according to various embodiments. It is understood that the flowchart of FIG. 2 provides merely an example of the many different types of functional arrangements that may be employed to implement the operation of the portion of the provider application 181 as described herein. As an alternative, the flowchart of FIG. 2 may be viewed as depicting an example of elements of a method implemented in the provider computing device 106 (FIG. 1A) according to one or more embodiments.

Beginning with box 203, the provider application 181 authenticates a wound care provider user. For example, the provider user may provide a username, a password, a numerical code, a one-time password, or other knowledge or possession tokens. Further, the provider user may be authenticated biometrically, including, for example, face recognition, fingerprint recognition, voice recognition, and/or other forms of biometric recognition.

In box 206, the provider application 181 receives patient information via a user interface 190 (FIG. 1A). For example, the provider user may enter various items of patient data 121 (FIG. 1A), including patent name, identifying number, address, insurance information 145 (FIG. 1A), shipping information 157 (FIG. 1A), compliance information 160 (FIG. 1A), health information 163 (FIG. 1A), and/or other information. In some cases, the relevant patient data 121 may be preexisting in the data store 112 or may be imported electronically from a medical record management system. In such cases, the provider user may provide, for example, a last name and birthdate (or other identifier), and the relevant information will be filled in via the user interface 190 or via the backend for the provider application 181 or the wound care management service 118 (FIG. 1A).

In box 209, the provider application 181 receives prescription information 142 (FIG. 1A) from the provider user via the user interface 190. For example, the provider user may supply a prescription name, dose, method of delivery, refill information, etc. Some or all of the information may be automatically populated from previous prescriptions. It is noted that the provider user may supply the information in boxes 206 and 209 via a speech-based user interface 190. The provider may also fill in an electronic signature to authorize the prescription.

In box 212, the provider user can use the camera 175 (FIG. 1A) of the provider computing device 106 to capture one or more wound images 148 of the patient's wound. Rather than using a separate camera application, the wound images 148 may be captured within the same provider application 181, so as to separate and classify the wound images 148 separately from other images or photos taken by the provider computing device 106. For example, the provider computing device 106 may be a smartphone, and the camera 175 may be an integrated smartphone camera. The wound images 148 may be captured simply to document the appearance of the wound or may be taken along a specified trajectory in order to automatically measure a dimension of the wound (e.g., length, width, and depth) or to generate a wound model 154 (FIG. 1A). For example, the provider application 181 may have a virtual ruler on the user interface 190 and by moving the camera 175 from side to side, the virtual ruler can measure a dimension of the wound.

In box 215, the provider application 181 determines one or more measurements of the wound. The provider user may manually enter these measurements, or these measurements may be determined using a “ruler” feature involving taking multiple wound images 148 or a video along a specified trajectory. In some cases, the wound measurements may be automatically determined using an analysis of the wound. For example, past wounds having known wound dimensions 151 may be analyzed and used to generate a ruleset that can estimate wound dimensions 151 without a specific measurement. This approach may be favored in lieu of an invasive measurement of a wound's depth using a depth gauge.

In box 218, the provider application 181 sends one or more of the wound images 148, the measurement(s), the patient data 121, and the prescription information 142 to the wound care management service 118. In some embodiments, the measurement(s) may be determined by processing performed by the wound care management service 118 in lieu of processing performed in the provider computing device 106.

In box 221, the provider application 181 may receive and present a result of a verification of the prescription information 142 via a user interface 190. For example, the wound care management service 118 may analyze the patient data 121 and determine that the prescription may not be accurate based on a machine learning analysis of past prescriptions and wound images. Alternatively, a pharmacy user may manually examine the prescription information 142 and the wound images 148 and determine that the prescription information 142 should be modified. As part of the verification, the inventory management service 115 (FIG. 1A) may be consulted and availability of the prescribed product in the pharmacy inventory data 139 (FIG. 1A) may be confirmed. The provider user may modify, cancel, or approve the prescription information 142 via the user interface 190 after receiving notification of the verification issue. Confirmation of prescription fulfillment and shipping may be provided to the provider user via the provider application 181. Thereafter, the operation of the portion of the provider application 181 ends.

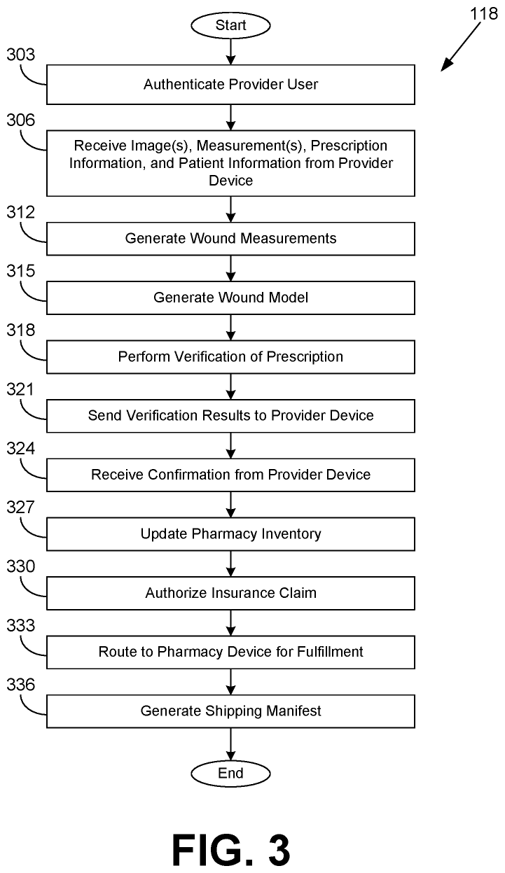

Turning now to FIG. 3, shown is a flowchart that provides one example of the operation of a portion of the wound care management service 118 relating to receiving and fulfilling prescriptions according to various embodiments. It is understood that the flowchart of FIG. 3 provides merely an example of the many different types of functional arrangements that may be employed to implement the operation of the portion of the wound care management service 118 as described herein. As an alternative, the flowchart of FIG. 3 may be viewed as depicting an example of elements of a method implemented in the computing environment 103 (FIG. 1A) according to one or more embodiments.

Beginning with box 303, the wound care management service 118 authenticates a provider user via a provider application 181 executed on a provider computing device 106 (FIG. 1A). For example, the provider user may supply a username, password, key, numerical code, biometric identifiers, and/or other security credentials that can be used to verify his or her identity. In box 306, the wound care management service 118 may receive one or more wound images 148 (FIG. 1A), wound measurements, prescription information 142 (FIG. 1A), and other patient data 121 (FIG. 1A) from the provider computing device 106. In some cases, the wound measurements may be automatically determined by the wound care management service 118 rather than included in the information supplied by the provider computing device 106.

At box 312, the wound care management service 118 may generate wound measurements through an automated analysis of the wound images 148. The measurements may be stored in the wound dimensions 151 (FIG. 1A). In box 315, the wound care management service 118 may generate a wound model 154 (FIG. 1A) from the wound images 148. For example, the wound care management service 118 may analyze the wound images 148 and generate a three-dimensional model of the wound that can be rotated, zoomed, or otherwise manipulated.

In box 318, the wound care management service 118 performs a verification on the prescription information 142. To this end, the wound care management service 118 may verify, using the prescription verification ruleset 127, that the prescription correctly corresponds to the type of wound seen in the wound images 148, spanning the wound dimensions 151, or shown in the wound model 154. For example, the prescription may be for an antibiotic that is unlikely to be effective for the severity of wound depicted in the wound images 148. Alternatively, the patient's health information 163 (FIG. 1A) may be associated with a different treatment modality, such as the use of a medicated foot bath in lieu of a topical ointment.

In box 321, the wound care management service 118 sends the results of the verification to the provider computing device 106. For example, verification and preauthorization results may be sent to a mobile device, along with a suggested change to the prescription. For example, the user interface 190 (FIG. 1A) of the provider application 181 may show a red “X” or some other symbol indicating a verification issue. The user interface 190 may also show detailed information as to why the prescription was not properly verified.

In box 324, the wound care management service 118 may receive a confirmation to proceed with the prescription from the provider user. For example, the provider user may disagree with or wish to overrule the verification problem identified by the wound care management service 118. Alternatively, the provider user may alter or modify the prescription information 142 or other information in order to address the identified verification problem.

In box 327, the wound care management service 118 communicates with the inventory management service 115 to verify that inventory to fulfill the prescription exists in the pharmacy inventory data 139 (FIG. 1A), and assuming the items are in the inventory, reserves that inventory for fulfillment of the prescription. The inventory management service 115 may coordinate placing orders for additional items as needed to maintain sufficient inventory to fulfill projected prescriptions.

In box 330, the wound care management service 118 may authorize an insurance claim by communicating with the insurance system 166 via the network 109. The wound care management service 118 may authorize the claim by presenting information about the prescription and the patient, including insurance information 145 (FIG. 1A). In addition, preauthorization may occur as part of the verification procedure in box 318.

In box 333, the wound care management service 118 routes the prescription to a pharmacy application 184 (FIG. 1A) executed on a pharmacy computing device 107 (FIG. 1A) for fulfillment. For example, the pharmacy application 184 may present a user interface 190 that is a dashboard showing pending prescriptions to be fulfilled. In addition, the user interface 190 may permit the pharmacy user to interact with the wound models 154, view the wound images 148, and see the wound dimensions 151. The user interface 190 may facilitate electronic communication between the pharmacy user and the provider user and the patient. For example, upon viewing the wound images 148 and/or interacting with a rendering of the wound model 154, the pharmacy user may wish to discuss the prescription with the provider user and/or the patient.

In box 336, in advance, or upon approval by the pharmacy user, the wound care management service 118 generates a shipping manifest with the shipping system 169 (FIG. 1A) via the network 109. In so doing, the wound care management service 118 uses the prescription shipping ruleset 124 (FIG. 1A) applied to the prescription information 142 to select the appropriate shipping carrier and shipping options. The wound care management service 118 may send a notification to the patient indicating the fulfillment status of the prescription. Thereafter, the operation of the portion of the wound care management service 118 ends.

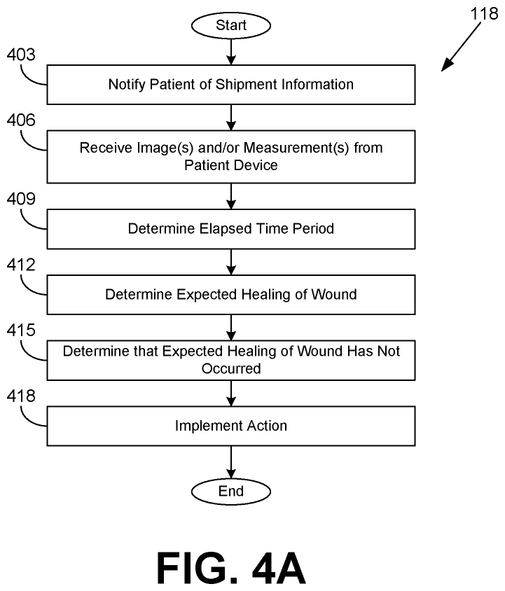

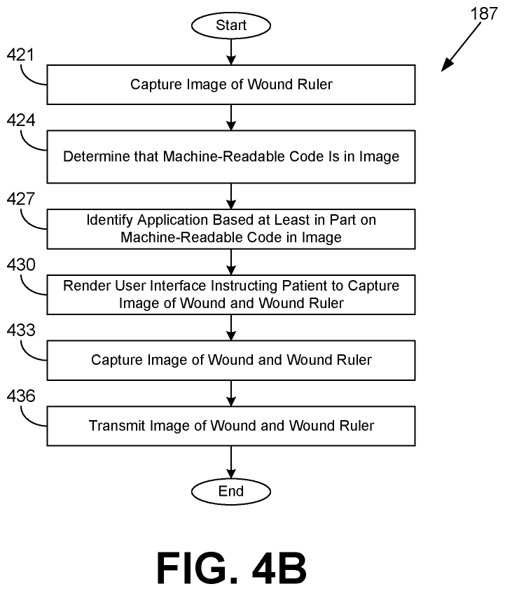

Moving on to FIG. 4A, shown is a flowchart that provides one example of the operation of a portion of the wound care management service 118 relating to patient healing and compliance according to various embodiments. It is understood that the flowchart of FIG. 4A provides merely an example of the many different types of functional arrangements that may be employed to implement the operation of the portion of the wound care management service 118 as described herein. As an alternative, the flowchart of FIG. 4A may be viewed as depicting an example of elements of a method implemented in the computing environment 103 (FIG. 1A) according to one or more embodiments.

Beginning with box 403, the wound care management service 118 notifies a patient via a patient computing device 108 (FIG. 1A) of shipment information relating to a fulfilled prescription. For example, the notification may include tracking information, expected delivery times, prescription use or handling instructions, and information on how to talk with a pharmacist.

In box 406, the wound care management service 118 receives one or more wound images 148 (FIG. 1A) and/or measurements from the patient computing device 108. For example, the patient may be directed to take pictures of the wound using their camera 175 (FIG. 1A) of their patient computing device 108 at specified intervals in order to judge expected healing. The images may be captured via the patient application 187 (FIG. 1A). The patient may be notified via a device notification, text message, email message, phone call, etc., that a new wound image 148 should be taken. In particular, the wound care management service 118 may send a compliance verification request to the patient requesting that use of the prescription corresponding to the prescription information be verified.

In box 409, the wound care management service 118 determines an elapsed time period since a previous wound image 148 was taken. For example, a previous wound image 148 may have been captured during a visit by the patient to the wound care provider, and one week may have elapsed.

In box 412, the wound care management service 118 determines the expected healing of the wound that should have occurred between the previous wound image(s) 148 and the subsequent wound images 148. This may be accomplished by analyzing the images and/or associated wound models 154 (FIG. 1A) using the wound healing ruleset 130 (FIG. 1A) constructed via machine learning of wound healing outcomes. In the image analysis, the wound care management service 118 may extract a baseline parameter from a previous wound image 148, determine an expected change to the baseline parameter based at least in part on a rule generated from an analysis of patient wound images taken over the time period, extract an updated parameter from a subsequent wound image 148, and then determine that a difference between the baseline parameter and the updated parameter is less than the expected change. For example, the parameters may relate to apparent wound size, wound color, and/or other characteristics. The expected healing may also be based on patient data 121 (FIG. 1A) such as the health information 163 (FIG. 1A) including health characteristics of the patient. For example, some patients may be expected to heal faster than others due to systematic conditions, age, and/or other factors.

In box 415, the wound care management service 118 may determine that the expected healing of the wound has not occurred. For example, the wound might not have improved or may even be worse. In box 418, the wound care management service 118 implements one or more actions in response to determining that the expected healing has not occurred. These actions may include notifying a pharmacy user via the pharmacy application 184 (FIG. 1A), notifying a provider user via the provider application 181 (FIG. 1A), implementing a refill of an existing prescription, suggesting a change to a prescription, and/or other actions. Thereafter, the operation of the portion of the wound care management service 118 ends.

Moving on to FIG. 4B, shown is a flowchart that provides one example of the operation of a portion of the patient application 187 according to various embodiments. It is understood that the flowchart of FIG. 4B provides merely an example of the many different types of functional arrangements that may be employed to implement the operation of the portion of the patient application 187 as described herein. As an alternative, the flowchart of FIG. 4B may be viewed as depicting an example of elements of a method implemented in the patient computing device 108 (FIG. 1A) according to one or more embodiments.

Beginning with box 421, the patient application 187 captures an image of a wound ruler 191 (FIG. 1B). In some cases, a user may use a camera application to capture the image using the camera 175 (FIG. 1A) of the patient computing device 108. In box 424, the patient application 187 determines that a machine-readable code 193 (FIG. 1B) is depicted in the image that has been captured. For example, the machine-readable code 193 may correspond to a QR code, a two-dimensional barcode, a three-dimensional barcode, or another code. The machine-readable code 193 may encode a uniform resource locator or URL, which may cause the patient application 187 to be identified, or may cause a particular website to be launched.

In box 427, the patient application 187 identifies an application based at least in part on the machine readable code 193 detected in the image. For example, the patient application 187 may decode the URL embedded within the machine-readable code, which then signifies the application. In one example, the application that is identified is a text messaging application.

In box 430, the patient application 187 renders a user interface instructing the patient to capture an image of the wound that also contains the wound ruler 191 for scale purposes. For example, the patient application 187 may open up a text messaging thread between the patient and the pharmacy or the provider, which automatically displays instructions to capture the image of the wound and the wound ruler 191.

In box 433, the patient application 187 captures the image of the wound and the wound ruler 191. In box 436, the patient application 187 transmits the image of the wound and the wound ruler 191 to a server. For example, the image may be transmitted to the provider application 181 (FIG. 1A), the pharmacy application 184 (FIG. 1A), or to the wound care management service 118 (FIG. 1A). The image may then be filed with the patient's profile as a wound image 148 (FIG. 1A) in the patient data 121 (FIG. 1A).

An estimated length, an estimated width, and an estimated depth of the wound in the image is determined based at least in part on the wound ruler captured in the image. For example, an image analysis based upon a machine learning model may be performed to extract the scale from the numbers and gradations shown on the wound ruler 191. The machine learning model may be trained on a number of such images of wound rulers 191 and provided with manually measured values corresponding to what is shown in the images. Thus, the machine learning model through training is able to identify the measurement characteristics of the wounds. Thereafter, the operation of the portion of the patient application 187 ends.

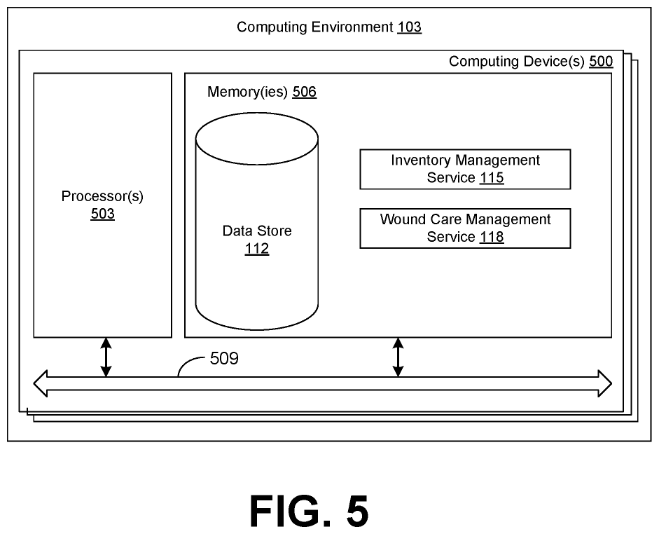

With reference to FIG. 5, shown is a schematic block diagram of the computing environment 103 according to an embodiment of the present disclosure. The computing environment 103 includes one or more computing devices 500. Each computing device 500 includes at least one processor circuit, for example, having a processor 503 and a memory 506, both of which are coupled to a local interface 509. To this end, each computing device 500 may comprise, for example, at least one server computer or like device. The local interface 509 may comprise, for example, a data bus with an accompanying address/control bus or other bus structure as can be appreciated.

Stored in the memory 506 are both data and several components that are executable by the processor 503. In particular, stored in the memory 506 and executable by the processor 503 are the inventory management service 115, the wound care management service 118, and potentially other applications. Also stored in the memory 506 may be a data store 112 and other data. In addition, an operating system may be stored in the memory 506 and executable by the processor 503.

It is understood that there may be other applications that are stored in the memory 506 and are executable by the processor 503 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 506 and are executable by the processor 503. In this respect, the term “executable” means a program file that is in a form that can ultimately be run by the processor 503. Examples of executable programs may be, for example, a compiled program that can be translated into machine code in a format that can be loaded into a random access portion of the memory 506 and run by the processor 503, source code that may be expressed in proper format such as object code that is capable of being loaded into a random access portion of the memory 506 and executed by the processor 503, or source code that may be interpreted by another executable program to generate instructions in a random access portion of the memory 506 to be executed by the processor 503, etc. An executable program may be stored in any portion or component of the memory 506 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 506 is defined herein as including both volatile and nonvolatile memory and data storage components. Volatile components are those that do not retain data values upon loss of power. Nonvolatile components are those that retain data upon a loss of power. Thus, the memory 506 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 503 may represent multiple processors 503 and/or multiple processor cores and the memory 506 may represent multiple memories 506 that operate in parallel processing circuits, respectively. In such a case, the local interface 509 may be an appropriate network that facilitates communication between any two of the multiple processors 503, between any processor 503 and any of the memories 506, or between any two of the memories 506, etc. The local interface 509 may comprise additional systems designed to coordinate this communication, including, for example, performing load balancing. The processor 503 may be of electrical or of some other available construction.

Although the inventory management service 115, the wound care management service 118, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowcharts of FIGS. 2-4 show the functionality and operation of an implementation of portions of the wound care management service 118, the provider application 181, the pharmacy application 184, and the patient application 187. If embodied in software, each block may represent a module, segment, or portion of code that comprises program instructions to implement the specified logical function(s). The program instructions may be embodied in the form of source code that comprises human-readable statements written in a programming language or machine code that comprises numerical instructions recognizable by a suitable execution system such as a processor 503 in a computer system or other system. The machine code may be converted from the source code, etc. If embodied in hardware, each block may represent a circuit or a number of interconnected circuits to implement the specified logical function(s).

Although the flowcharts of FIGS. 2-4 show a specific order of execution, it is understood that the order of execution may differ from that which is depicted. For example, the order of execution of two or more blocks may be scrambled relative to the order shown. Also, two or more blocks shown in succession in FIGS. 2-4 may be executed concurrently or with partial concurrence. Further, in some embodiments, one or more of the blocks shown in FIGS. 2-4 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein, including the inventory management service 115 and the wound care management service 118, that comprises software or code can be embodied in any non-transitory computer-readable medium for use by or in connection with an instruction execution system such as, for example, a processor 503 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the inventory management service 115 and the wound care management service 118, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device 500, or in multiple computing devices 500 in the same computing environment 103.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A system, comprising:
at least one computing device; and
at least one application executable in the at least one computing device, wherein when executed the at least one application causes the at least one computing device to at least:
generate a prescription verification ruleset by processing a set of past prescription information and a set of past wound images using a machine learning approach;
receive at least one image of a wound from a mobile device, the at least one image of the wound including a wound ruler displaying a machine-readable code;
receive prescription information from the mobile device, the prescription information comprising a medication;
perform a verification on the prescription information based at least in part on a machine learning analysis of the at least one image using the prescription verification ruleset;
obtain a preauthorization for the medication from an insurance provider;
send the verification results and the preauthorization results to the mobile device;
receive a confirmation to fulfill the medication from the mobile device; and
send the prescription information to a pharmacy device for fulfillment.

2. The system of claim 1, wherein the verification confirms whether the prescription information corresponds to the wound depicted in the at least one image.

3. The system of claim 1, wherein the machine learning analysis extracts at least one parameter characterizing the wound from the at least one image, and the verification applies at least one rule generated from the past prescriptions associated with the at least one parameter from the prescription verification ruleset.

4. The system of claim 1, wherein when executed the at least one application further causes the at least one computing device to at least generate shipping requirements for a pharmaceutical product described in the prescription information.

5. The system of claim 1, wherein when executed the at least one application further causes the at least one computing device to at least generate a three-dimensional model of the wound based at least in part on the at least one image, the three-dimensional model being displayed in a user interface.

6. The system of claim 1, wherein when executed the at least one application further causes the at least one computing device to at least:
receive at least one subsequent image of the wound from a patient mobile device;
determine a time period that has elapsed between the at least one image and the at least one subsequent image;
determine by a comparison of the at least one subsequent image to the at least one image that the wound has not experienced an expected healing over the time period; and
implement at least one action in response to determining that the wound has not experienced the expected healing over the time period.

7. The system of claim 1, wherein when executed the at least one application further causes the at least one computing device to at least send a notification to a patient indicating a fulfillment status of a prescription corresponding to the prescription information.

8. The system of claim 1, wherein when executed the at least one application further causes the at least one computing device to at least send a compliance verification request to a patient requesting that use of a prescription corresponding to the prescription information be verified.

9. A method, comprising:
generating a prescription verification ruleset by processing a set of past prescription information and a set of past wound images using a machine learning approach;
capturing at least one image of a wound via a camera of a mobile device, the at least one image of the wound including a wound ruler displaying a machine-readable code;
determining at least one measurement of the wound by analyzing the at least one image;
receiving prescription information for treating the wound via a user interface rendered by the mobile device, the prescription information comprising a medication;
verifying the prescription information based at least in part on a machine learning analysis of the at least one image using the prescription verification ruleset;
obtaining a preauthorization for the medication from an insurance provider;
sending the verification results and the preauthorization results to the mobile device;
receiving a confirmation to fulfill the medication from the mobile device; and
sending the at least one image, the at least one measurement, and the prescription information to a pharmacy computing device via a network.

10. The method of claim 9, wherein determining at least one measurement of the wound is further based at least in part on a rule generated from a set of wound images having known measurements.

11. The method of claim 9, wherein the at least one measurement includes a depth of the wound.

12. The method of claim 9, further comprising:
receiving a three-dimensional model of the wound from the pharmacy computing device, the three-dimensional model being generated based at least in part on the at least one image; and
rendering the three-dimensional model for display by the mobile device.

13. The method of claim 9, wherein the at least one image comprises a video, and the method further comprises directing a user of the mobile device to capture the video of the wound by moving the camera of the mobile device about the wound along a particular trajectory.

14. The method of claim 9, further comprising:
determining that a verification issue exists based at least in part on a comparison of the at least one image of the wound and a machine learning analysis of a plurality of wound images and past prescriptions

15. A method, comprising:
receiving a first image via a patient mobile device;
determining that the first image includes a machine-readable code, the machine-readable code being displayed on a wound ruler;
identifying an application based at least in part on data encoded in the machine-readable code;
instructing a user to capture a second image of a wound and the wound ruler via a user interface;
receiving the second image from the patient mobile device; and
transmitting the second image to at least one other computing device for analysis.

16. The method of claim 15, further comprising determining an estimated length, an estimated width, and an estimated depth of the wound in the second image based at least in part on the wound ruler captured in the second image.

17. The method of claim 15, wherein the application is a text messaging application, and the user interface includes a text messaging thread with at least one of: a pharmacy computing device or a provider computing device.

18. The method of claim 15, further comprising automatically determining by the comparison of the second image to a third image previously taken of the wound that the wound has not experienced an expected healing over a time period.

19. The method of claim 18, wherein determining by the comparison of the second image to the third image that the wound has not experienced an expected healing over the time period further comprises:
extracting a baseline parameter from the second image;
determining an expected change to the baseline parameter based at least in part on a rule generated from an analysis of patient wound images taken over the time period;
extracting an updated parameter from the third image; and
determining that a difference between the baseline parameter and the updated parameter is less than the expected change.

20. The method of claim 18, wherein the expected healing over the time period is determined by a wound healing ruleset constructed by a machine learning algorithm that analyzes wound images with the wound healing outcomes.
